(19) United States
(12) Patent Application Publication
(10) Pub. No.: US 2016/0018100 A1
(43) Pub. Date: Jan. 21, 2016

(54) COMPACT STEAMER

(71) Applicants: Jerod Batt, Cincinnati, OH (US); Jainagesh Sekhar, Cincinnati, OH (US); Ramgopal Vissa, Hyderbad (IN); Michael Connelly, Cincinnati, OH (US); Brian Kandell, Cincinnati, OH (US)

(72) Inventors: Jerod Batt, Cincinnati, OH (US); Jainagesh Sekhar, Cincinnati, OH (US); Ramgopal Vissa, Hyderbad (IN); Michael Connelly, Cincinnati, OH (US); Brian Kandell, Cincinnati, OH (US)

(73) Assignee: MICROPYRETICS HEATERS INTERNATIONAL, INC., Cincinnati, OH (US)

(21) Appl. No.: 14/114,542

(22) PCT Filed: Jun. 8, 2012
(86) PCT No.: PCT/US12/41460

§ 371 Date: Oct. 29, 2013

Related U.S. Application Data
(60) Provisional application No. 61/497,590, filed on Jun. 16, 2011.
(60) Provisional application No. 61/555,053, filed on Nov. 3, 2011.

(51) Int. Cl. F22B 1/28; A61L 2/20

(52) U.S. Cl. CPC F22B 1/284; A61L 2/20

(57) ABSTRACT

A compact steaming device, which may be handheld, and method for the generation at one atmosphere and projection of superheated steam over objects and surfaces for the elimination and control of unwanted microorganisms and pests including, but not limited to bedbugs, fleas, ants, lice and mites. A preferred embodiment of such a compact device comprises a vessel, a base, heating coils, a superheated steam generator and an external power supply for the initial boiling of a liquid to vapor and for subsequent heating of the vapor to superheated temperatures. The device and method allow for the generation of steam or gas and subsequent superheating to be accomplished safely at one atmosphere. The preferred embodiment allows for the free flow of the gas through and out of the compact device safely and effectively relieving any resulting pressure. A multi-watt embodiment is also envisioned which may be handheld or stationary.

### CROSS REFERENCE TO RELATED APPLICATIONS

This patent application claims the benefit of provisional patent applications Ser. No. 61/497,590, filed Jun. 16, 2011 and Ser. No. 61/555,053, filed on Nov. 3, 2011 by the present applicants and utilizes features disclosed in U.S. patent application Ser. No. 11/682,107 filed on Mar. 5, 2007, as well as PCT Application Nos. PCT/US07/84670, entitled “Heating and Sterilizing Apparatus and Method of Using Same” filed on Nov. 14, 2007, and PCT/US12/25334, entitled “Pressurized Point-Of-Use Superheated Steam Generation Apparatus And Method” filed on Feb. 16, 2012 also filed by the present applicant, the disclosures of which are hereby incorporated by reference herein in their entirety

### BACKGROUND

1. Field

This application relates to a compact steamer apparatus and method for control and elimination of unwanted organisms such as, but not limited to mites, fleas, ants, bedbugs and microorganisms utilizing the unique properties of superheated steam.

2. Prior Art

The prior art does not contain examples of an apparatus which combines the necessary and desired features for effective, economical and widely applicable and adaptable elimination of unwanted insects and other organisms. Superheated steam provides the necessary temperature needed to kill insects such as mites, fleas and bedbugs and their eggs. The unique properties of superheated steam allow for great heat to be applied (250° C.) with minimal disturbance caused by wetness or humidity. Devices exist that produce superheated steam, but they are often large, bulky and complicated and are difficult if not impossible to use in close quarters. A compact superheated steaming device is needed that is compact enough to allow access to most spaces where household or other pests live or hide.

Compact and handheld steamers presently exist, but they often do not produce superheated steam. Some are very complicated making them expensive. Others need to be in contact to a surface for the direct application of steam thereby rendering them less useful. These are often intended for a specific purpose, such as the removal of wrinkles in cloth, and are not applicable to pest control. The water source is often remote to the steamers making them more bulky and less portable.

The prior art contains examples which disclose devices utilizing steam, sometimes superheated, to destroy insects and various other pests. U.S. Pat. No. 7,797,878 to Schuster (2010) presents a device to suppress fire ants utilizing the injection of superheated steam into an ant colony. This invention has several possible limitations. It is heavy and bulky and relies upon a handcart for ease of movement. The generation of superheated steam uses a complicated system to create steam and then reheat it to a superheated state. This invention only performs its intended purpose when a probe is driven into the ground to reach insects.

Likewise U.S. patent application Ser. No. 12/757,969 published as 2011/0041782 by Vaughan presents a device to control pests and weeds utilizing steam, sometimes superheated, and hot air. This system is much more bulky than the present apparatus requiring a cart with wheels to transport it. It is not compact and its usage in tight areas would be very limited. The system is complicated needing a burner to heat water into steam, an electric blower to move the air and a means to pump the needed water. Associated controls and circuitry are needed as well.

U.S. Pat. No. 5,867,935 (1999) and U.S. Pat. No. 5,848,492 (1998) both to Brown present an invention intended to apply superheated steam in agricultural applications to defoliate and to eliminate insect pests. The major drawback of this apparatus is its extreme bulkiness, heaviness and size. One embodiment is equipped with wheels so that it may be pulled by a tractor or other means into position. It can only be utilized outdoors or where there is a lot of space, thereby limiting its usefulness. Indoor use is limited if not possible in most situations. The system used for the heating and the projection of the steam is complicated as well.

U.S. Pat. No. 5,378,086 to Campbell, Jr. (1995) can utilize superheated steam but is not portable but is instead a permanent underground system for pest extermination. U.S. Pat. No. 4,620,388 (1986) and U.S. Pat. No. 4,716,676 (1988) both to Imagawa are large stationary systems for the elimination of pests on or in fruit which is placed inside of the invention. These three inventions have very limited, specific and non-portable intended applications.

U.S. Pat. No. 4,756,118 to Evans, II (1988) has a handheld applicator, but has a fuel and gas source that both are external to the applicator making it bulky and complicated. Gas is vaporized by a flame and then injected into fire ant colony. It is not intended to be used in any other manner, thereby limiting possible applications.

U.S. Pat. No. 4,637,161 to Turner (1987) and U.S. Pat. No. 7,752,802 to McDonald (2010) are intended for underground elimination of ants and must be set up, stuck in the ground and used in place. They are thus not handheld and are limited in their usage.

U.S. Pat. No. 7,155,117 to Leung et al. (2006), U.S. Pat. No. 3,695,066 to Doyel (1972) and U.S. application Ser. No. 12/341,614 published as 2009/0313767 by Tanner et al. are handheld steam generators designed generally for the removal of wrinkles from fabric or clothing. Each is fairly complicated either electronically or mechanically and none is designed to produce superheated steam. These inventions are meant to be in close physical contact with a work-piece and will not function effectively if not. Usefulness for other purposes is thereby greatly diminished.

There is a great need for a device which can effectively and conveniently destroy and control insect infestations. Desirable qualities in such a device would be effectiveness, compactness, ease of use, simplicity, having a self-contained water supply and a convenient means to access external electric power if not an internal power supply. A simple compact handheld superheated steamer with a self-contained water supply would meet these requirements. Such a device would be useful in the home and in places of business to remove unwanted insects such as dust mites and roaches and fleas and ticks brought in by household pets. The increase in reported cases of bedbug infestations, caused partially by the banning of DDT and other pesticides, could be countered by the use of such a steamer that would eliminate the bedbugs and their eggs in their indoor habitat with little disturbance. It is anticipated that such device could control these and other insects and their eggs as well as insects or other organisms in dormant stages.

### SUMMARY

In accordance with an exemplary embodiment, a compact steamer, which may be handheld, comprises a vessel having a base containing an arrangement of electrically charged heating elements for the heating of a liquid into a gaseous state and a superheated steam generator for the heating of the gas into a superheated condition and the gases subsequent application to objects and surfaces all done at one atmosphere.

More particularly, with the compact steamer, heat can be utilized, as a means to control and destroy living or non-living organisms including, but not limited to, ants, mites, fleas, parasites, bed bugs, other insects (bugs) and microorganisms without disturbing or altering the surrounding area. Such utilization of heat for these purposes in a compact handheld design is not found in the prior art Improvements to the prior art that are needed can be provided by the application of superheated steam in the manner presented in this application. The unique characteristics of superheated steam allow for its application to surfaces, items and locations where such household pests may be found without removal or damage of such items. Superheated steam has high enthalpy at a relatively low temperature and also low specific volume. This system allows a gas, or steam, to be in a phase separating meta-stable state, allowing for a gas, or steam, that is hot enough to destroy these targeted organisms yet can dry rapidly, thereby minimizing damage to the surrounding environment. No relative prior art possesses a combination of the generation and application of superheated steam in a compact handheld design, used here for, but not limited to, the elimination of household pests and microorganisms.

### DRAWINGS—FIGURES

FIG. 1 shows an exemplary embodiment of the compact steamer.

FIG. 2 shows a schematic of an embodiment of the electrical circuit design of the compact steamer.

FIG. 3 shows a schematic of an embodiment of the base heating coil layout of the compact steamer.

FIG. 4 is an embodiment of the heater contained within the superheated steam generator of the present application.

FIG. 5 is an exploded view of the heater contained within the superheated steam generator.

FIG. 6 is a cross sectional view of the heater contained within the superheated steam generator.

FIG. 7 is a view of the heating coils contained within the heater.

FIG. 8 is an embodiment of the superheated steam generator utilized in the present application.

FIG. 9 is a further embodiment of the superheated steam generator utilized in the present application.

FIG. 10 is a cross-sectional view of an exemplary embodiment of the compact steamer.

FIG. 11 shows a further embodiment of the compact steamer.

FIG. 12 shows the further embodiment of the compact steamer with a transparent view of its outer shell.

FIG. 13 shows the further embodiment of the superheated steam generator without its outer shell.

FIG. 14 shows the further embodiment of the superheated steam generator without its outer shell and with a transparent view of its steam vessel.

FIG. 15 shows the further embodiment of the superheated steam generator without its outer shell, steam vessel, frame and shroud.

FIG. 16 is a view of the supercharger.

FIG. 17 is a cut away view of the supercharger without a tubular housing.

FIG. 18 is a cut away view of the supercharger without a tubular housing and without two sections of spacers.

FIG. 19 is a cut away view of the supercharger without a tubular housing and without all spacers showing the internal arrangement of the heating coils.

FIG. 20 is a cut away view of the supercharger without a tubular housing and without all spacers showing the internal arrangement of the heating coils.

FIG. 21 is a cut away view of the supercharger without a tubular housing and without all spacers showing the internal arrangement of the heating coils.

FIG. 22 is an overall view of a multi-supercharger steam heater.

FIG. 23 is a view of the multi-supercharger steam heater without its lid showing the water chamber and the superchargers positioned within

FIG. 24 is a view of the multi-supercharger steam heater without its lid and upper body showing the superchargers and the heating coil.

FIG. 25 is a view of the multi-supercharger steam heater without its lid, upper body and lower body showing the superchargers, heating coil and steam chamber.

DRAWINGS - Reference Numerals

| | | | |
|---|---|---|---|
| 1. | compact steamer embodiment | 2. | steam vessel |
| 3. | base | 4. | heating coil |
| 5. | fill opening | 6. | handle |
| 7. | screw cap | 8. | head |
| 9. | neck | 10. | industrial gas heater |
| 11. | superheated steam | 12. | cylindrical tubular housing |
| | generator housing | | |
| 13. | steam return | 14. | gas entry port |
| 16. | gas exit port | | |
| 18. | open end | 20. | end cap |
| 22. | annular sidewall | 24. | end wall |
| 26. | stepped passage | 28. | inner helical coil |
| 28a. | generally continuous wire | 28b. | gap |
| 28c. | adjacent turn | 28d. | terminal lead wire |
| 28e. | flow path | 28f. | bare wire cross section |
| 30. | outer helical coil | 30a. | generally continuous wire |
| 30b. | gap | 30c. | adjacent turn |
| 30d. | terminal lead wire | 30e. | flow path |
| 30f. | bare wire cross section | 34. | spacer |
| 100. | superheated steam generator | | |
| 200. | superheated steam | 202. | gas inlet source |
| | generator embodiment | | |
| 204. | power cord grip | 206. | gas inlet |
| 208. | manifold housing | 210. | casing |
| 212. | delivery tube | 214. | end plate |
| 216. | fluid reservoir | 218. | feed line |
| 220. | needle valve | 222. | reactor vessel |
| 224. | porous medium | 226. | exit nozzle |
| 228. | diffuser | | |
| 300. | superheated steam | 302. | pump |
| | generator embodiment | | |
| 304. | fluid reservoir | 305. | valving |
| 306. | outer jacket housing | 308. | chamber |
| 310. | inlet | 312. | conduit |
| 314. | outlet | 405. | power switch |
| 410. | safety cut-off | 415. | heater switch |
| 430. | liquid level window | 500. | compact steamer |
| | | | embodiment |
| 505. | outer shell | 506. | concave base |
| 510. | cooling holes | 520. | diverter switch |
| 525. | diverter | 527. | direct steam line |
| 529. | diffused steam line | 530. | direct applicator |
| 540. | diffused applicator | 550. | frame |
| 560. | shroud | 562. | vessel bottom |
| 570. | fill level band | 575. | uniform space |
| 600. | supercharger | 602. | supercharger coil |
| 700. | multi-kW steamer | 705. | lid |
| 710. | upper body | 712. | lower body |
| 715. | water inlet | 720. | steam outlet |
| 725. | water chamber | 730. | steam chamber |

### DETAILED DESCRIPTION

In an exemplary embodiment of the present application a compact steam generator is envisioned consisting of a vessel for the generation of steam or gas and a superheated steam generator that will heat the steam or gas to superheated temperature levels. Features, sub-assemblies and aspects of the superheated steam generator will be described in detail below and are to be found as well in PCT application PCT/US07/84670 and U.S. patent application Ser. No. 11/682,107 filed by the present applicant. This exemplary embodiment will allow for the control, destruction and elimination of household pests and other living organisms including, but not limited to, ants, mites, dust mites, fleas, bed bugs, other insects and microorganisms. Most of such organisms will be destroyed at temperatures of 140° C., which is a temperature easily obtainable by this embodiment. Anticipated are compact steamers that will attain temperatures of over 120° C. or over 140° C. or over 160° C. Tests results indicate that this apparatus is capable of safely producing steam temperatures of 250° C. These temperatures will be reached at one atmosphere allowing for a safer superheated steam generating and producing apparatus with no hazardous high pressure. Only the initial generation of the steam will be contained in the vessel. Heating to superheated temperatures will be accomplished by a superheated steam generator in-line and point-of-use at one atmosphere. The characteristics of superheated steam allow for its application to surfaces, items and locations where such household pests may be found without removal or damage of such items. Such steam allows for the highest enthalpy for the lowest temperature and the lowest specific volume. This system allows a gas, or steam, to be in a phase separating meta-stable state, allowing for a gas, or steam, that is hot enough to destroy these targeted organisms yet can dry rapidly, thereby minimizing damage to the surrounding environment.

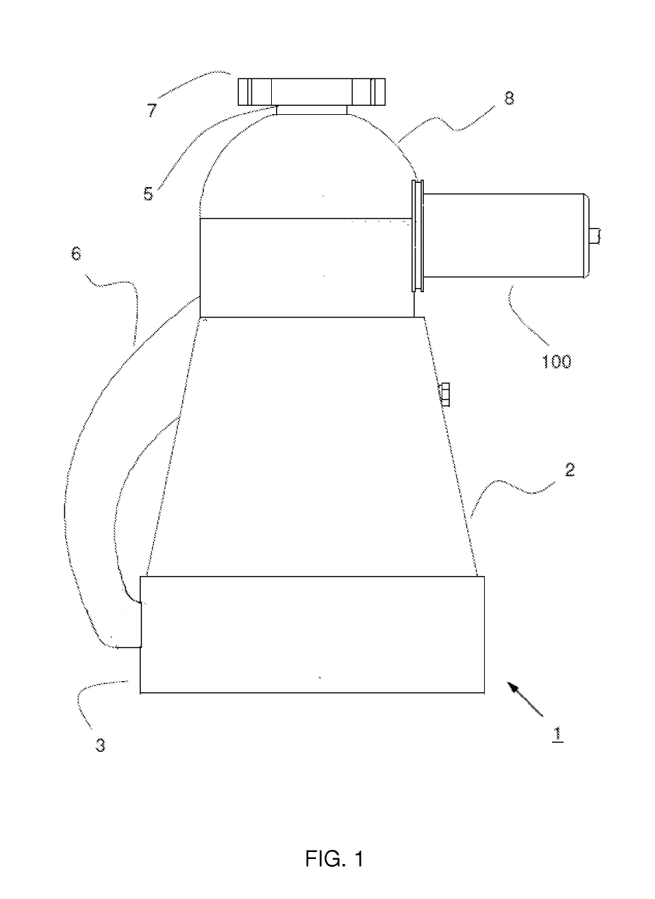

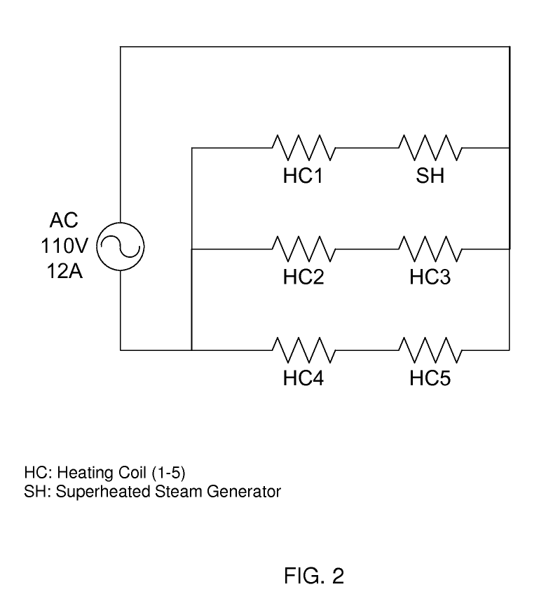

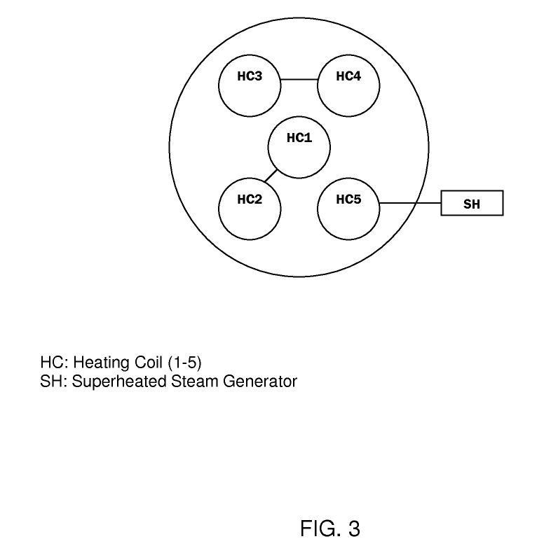

This exemplary embodiment consists, as shown above in FIG. 1 of a vessel in which water or other liquids are added. Such vessels may be coated with nanoscale anti-microbial coatings as suggested by PCT/US07/85564. Such coatings would be especially valuable in areas where water or humidity is present and where micro-organisms are common The coatings would control such growth. The vessel may be fitted with a sealable opening, such as a screw cap, for the addition of liquids, envisioned in this embodiment as being located near the top of the vessel. The vessel, in this embodiment, has a handle affixed to its side to allow for ease of grasping and manipulating the steamer by a user. The vessel is equipped with an electrical means for heating the contained liquid to its boiling point thus producing steam or gas. In this embodiment the electric power comes from an external 110V/12 A power, however it is envisioned that the electric power could come from a self contained and removable power pack or battery system. Heating coils are located in the base of the vessel to produce the heat. Other voltages, amperages and total power used in testing are listed below. Possible anticipated electrical circuit design and base heating coil layout are shown in FIGS. 2 and 3.

After the steam or gas is produced by the heat produced by the coils in the base of the vessel, the steam or gas freely flows up and into a superheated steam generator that is affixed to the vessel. The superheated steam generator is attached in a manner to allow free access of the steam from the vessel into the generator. Further refinements of this free access and attachment are contemplated as well. It is anticipated that the pressures developed in the vessel will generally be low and will be relieved upon the steam entering freely into and out of the superheated steam generator. In this embodiment the superheated stem generator is attached to the top of the vessel near the sealable opening, of fill cap, opposite the heating coils in the base of the vessel (FIG. 1). The steam is then superheated by these and flows out of the superheated steam generator for application. The pressure created in the vessel by the formation of the initial steam is the force that projects the superheated steam out of the steamer. Thus, the apparatus is very safe since pressure never builds up to a critical level, but instead is constantly released and kept to one atmosphere as the steam builds and flows freely out of the superheated steam generator.

Unlike prior art, the present apparatus has no internal fan for the projection or movement of the superheated steam. Such a fan is not needed as the pressure formed in the vessel will project the steam. Also, the present apparatus has no external source of steam or water and the tubing, lines or reservoirs associated with external sources. Here the liquid and steam sources are completely self-contained in the unit allowing for improved ease of use and enhanced safety. Only the electrical power to heat the coils in the vessel and the superheated steam generator are external to the present apparatus, but anticipated embodiments using integral power packs or batteries would eliminate such external needs. As anticipated, this apparatus needs only a 110V/12 A power outlet to plug into allowing for great convenience of use at the point of need wherever the application of superheated steam is necessary.

Other embodiments of the compact steamer are contemplated by the applicants that incorporate features which include, but are not limited to: External water source; Larger or smaller capacity vessel and materials out of which to construct the same (Micro-sized or Macro-sized); Varying configurations of the vessel and integral sealable opening and method to seal such opening; Fluid level gauge; Alternative base heating coil arrangements and power systems and requirements; different sizes and configurations of the superheated steam generator; Safety cut-off switch or valve; Mixing chamber; Flow control devices, valves or systems; Production of micro-steam or macro-steam; Boiling of fluids other than water to produce various gas other than steam; and the ability to includes additives in the water or fluid to produce desired chemically enhanced vapors.

### Operation

Operation of the exemplary embodiment is simple, safe and straightforward. Water or liquid is placed in the vessel which is, in essence, a self contained water supply. A sealable opening with a screw-cap is envisioned for the purpose of filling the steamer with water. The opening is then sealed to keep the steam from escaping and to assist in directing the steam to the superheated steam generator. The steamer is plugged into an external power supply which supplies electricity to the coils in the base of the vessel. The water then boils creating steam. Having free access to the superheated steam generator, the steam will flow into it and be heated by the generator and converted to superheated steam. The pressure developed in the vessel upon the initial heating of the water will be relieved by its free passage through the steam generator which is not sealed from the outside atmosphere. The pressure will also act as the means by which the superheated steam will be projected out of the steamer and on to surfaces where treatment is desired.

The steam is superheated as it is pushed, by pressure from the steam formation, through the superheated steam generator and can then be direct towards and upon any desired surface. The characteristics of superheated steam allow it to maintain a temperature that is effective in eliminating many unwanted living organisms, but also allows quick drying without residue and water damage. The compact steamer as contemplated in this exemplary embodiment is thus extremely useful in pest control as well as a host of other applications where superheated steam is needed. The compact design with internal water supply allows for the use of the steamer in areas and conditions without an external water source. Only an external power source is needed. An embodiment with an internal power source will make the steamer completely independent of external water or power. By using the pressure developed in the vessel to drive the steam through and out of the steamer the apparatus is mechanically and electrically more simple than the prior art. There are no moving parts, only a vessel, heating elements and a superheated steam generator.

FIG. 1 shows an exemplary embodiment of a compact steamer 1 that may be configured to be handheld. The steamer 1 comprises a bottle-shaped steam vessel 2, base 3, handle 6 and a superheated steam generator 100 contained partially with in the head 8. Water or other liquid is added into the vessel 1 through the fill opening 5 which is the upper end of a neck 9 and which will be sealable with a screw cap 7 or other means. The water will be heated to its boiling point in the vessel 1 by heating coils 4 located in the base 3. In this embodiment electrical power is brought to the coils from an outside source through an external power line (not depicted) which is plugged into a standard 110V/12 A outlet. The same power source will also provide electricity for the superheated steam generator 100. Alternate embodiments anticipate an internal source or electricity consisting of a removable or rechargeable battery system.

FIG. 2 shows an embodiment for the electrical circuit design for the compact steamer 1. An external AC 110V/12 A power source provides electricity for 3 parallel circuits of 110V/4 A each. This design anticipates 4 heating coils 4 connected in 2 pairs in series. A fifth heating coil 4 is connected in series to the superheated steam generator 100 by an electrical connection 8. The heating coils 4 and the superheated steam generator 100 are anticipated to run at 55V/4 A each. An anticipated layout of the heating coils 4 and the superheated steam generator 100 showing series connections between the pairs of coils 4 and the steam generator 100 is presented in FIG. 3.

As the water heats up in the steam vessel 2 and turns to steam, it rises through the neck 9 and flows freely into the steam generator 100 where it is superheated and expelled to the atmosphere. FIG. 1 shows that the steam generator 100 is located at the upper end of the steam vessel 2 opposite the base 3 connected to the steam vessel 2 by a neck 9. The neck 9 is hollow allowing free and unobstructed passage of steam or vapor from the steam vessel 2 into the super heated steam generator 100. The neck 9 also serves as the conduit through which liquid is supplied to the steam vessel 2. Generated pressure produced by the formation of steam is relieved by the passage of it through the neck 9 and out of the steam generator 100, thus making it safer than examples in the prior art.

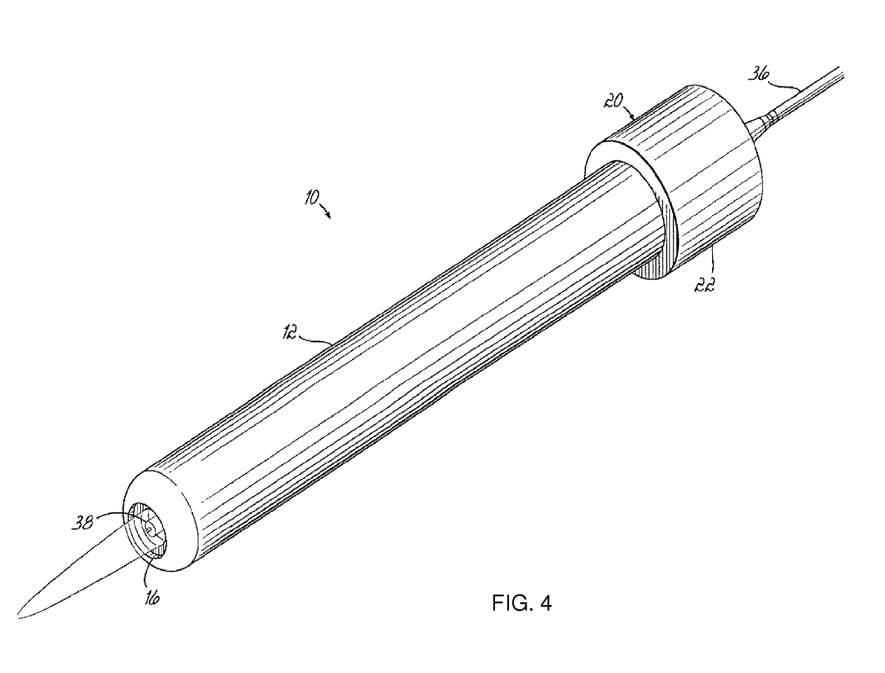

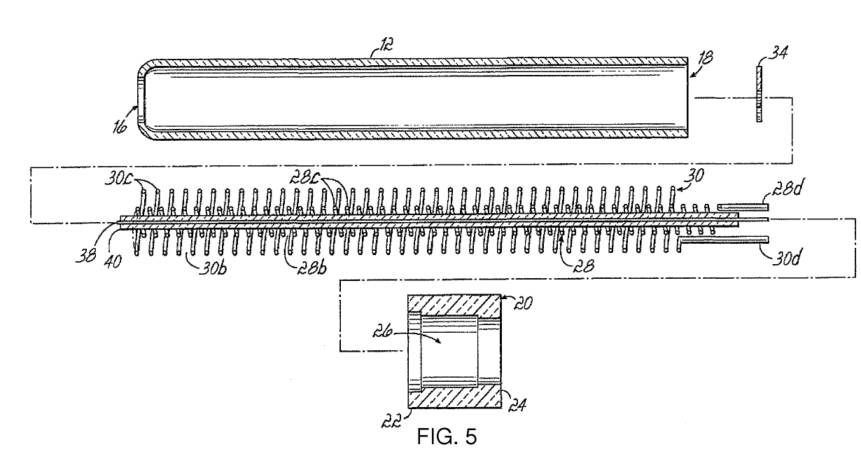

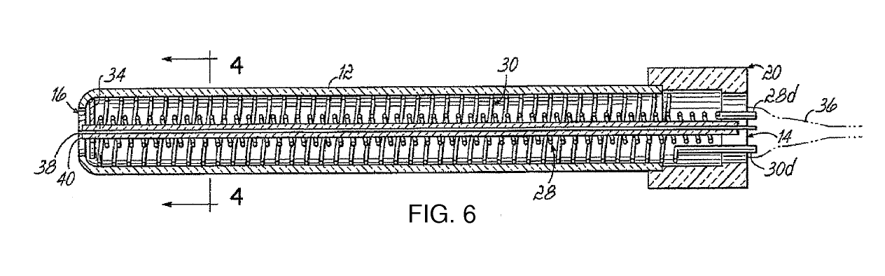

Referring to FIGS. 4-6, an exemplary embodiment of an industrial gas heater 10 according to this invention is shown. The heater 10 includes a generally right circular cylindrical tubular housing 12 having a gas entry port 14 at a first end of the housing 12 spaced from a gas exit port 16 at an opposite end of the housing 14. The housing 14 may be a monolithic ceramic tube or other material such as a metallic enclosure. However, we have found that the temperature of the gas heated within the assembly is increased anywhere from 25-200° C. when a ceramic housing is utilized.

The gas entry port 14 is proximate an open end 18 of the housing 14 and is selectively closed by an end cap 20 mounted on the open end 18 of the housing 14. The end cap 20 may be made from a ceramic of approximately 90 percent aluminum oxide. The cap 20 includes an annular sidewall 22 and an end wall 24. The end cap 20 is a partially open end cap and according to various embodiments of this invention, the end cap 20 can be fully or partially open with additional openings for electrical feed-throughs and thermocouple feed-throughs. A stepped passage 26 is formed on the interior of the sidewall 22 and the gas entry port 14 is on the end wall 24. The opening diameter of the gas entry port 14 to the gas exit port 16 may be at a ratio of about 2:1.

The gas heater 10 includes an inner helical coil 28 and an outer helical coil 30 contained within the tubular housing 12. The inner and outer coils 28, 30 are coaxially aligned and concentrically arranged as right circular helical coils within the housing 12 to define a substantially unobstructed annular space 32 for passage of gas through the housing 12 from the gas entry port 14 to the gas exit port 16. In one embodiment, each coil 28, 30 is formed from a generally continuous wire 28a, 30a, respectively, concentrically wound into right circular helical coils. A diameter of the wire 28a, 30a for each coil may range from about 0.1 mm to about 6 mm A gap 28b, 30b between the adjacent turns 28c, 30c of each coil 28, 30 may range from about 0.01 mm to about 85 mm The gap or pitch of each coil 28, 30 may increase adjacent to the entry port 14 and terminal lead wires 28d, 30d.

We have found that where the outer coil 30 is in close proximity to and/or in contact with the inside face of the tubular housing 12, the gas processed in the heater is heated approximately 25° to 200° C. higher than if the outer coil 30 is not in such a configuration relative to the housing 12. Additionally, a spacer 34 which may be ceramic is positioned at the distal end of the coils 28, 30 proximate the gas exit port 16. The spacer 34 increases the useful life of the coils 28, 30 and minimizes coil deformation over extended periods of use.

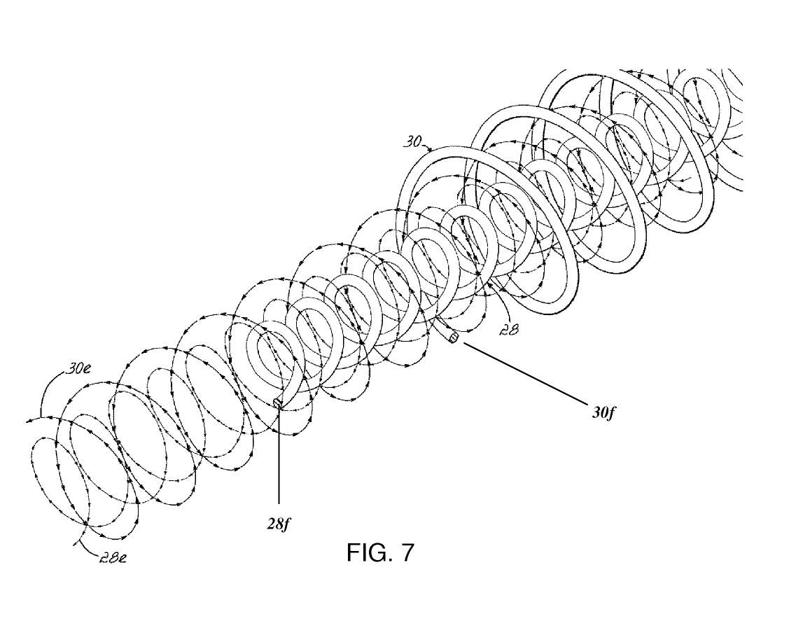

Among the advantages provided by a gas heater 10 according to this invention is the increased contact between the gas flowing from the entry port 14 to the exit port 16 with the coils 28, 30. For example, the coils 28, 30 may be similarly wound or wound in opposite directions as shown in FIG. 7. Gas flowing through the housing 12 passes between the coils 28 and 30. Additionally, gas flowing between the adjacent turns 28c, 30c of the respective coils 28, 30 flows in a riffling or spiraling configuration as schematically shown in FIG. 7 with flow paths 28e and 30e. The wire of the coils 28 and 30 are composed of bare wire which can be defined otherwise as having a solid or monolithic cross section or as being unclad or having no coating or insulation. Such composition is illustrated by wire cross sections 28f and 30f in FIG. 7. The coils 28 and 30 are wound in a round configuration as opposed to an oval or non-round shape found in the prior art. With the windings of the respective coils 28, 30 being in opposite direction, increased mixing of the gas with the coils 28, 30 is provided to obtain a more turbulent gas flow. This arrangement provides for increased thermal transfer from the heated coils 28, 30 to the gas relative to prior art industrial gas heating systems.

The range of gap spacing between the adjacent turns 28c, 30c of the wires 28a, 30a in the coils 28, 30 is between about 35 mm and about 85 mm with the presently preferred being about 40 mm for the inner coil 28 and about 65 mm for the outer coil 30.

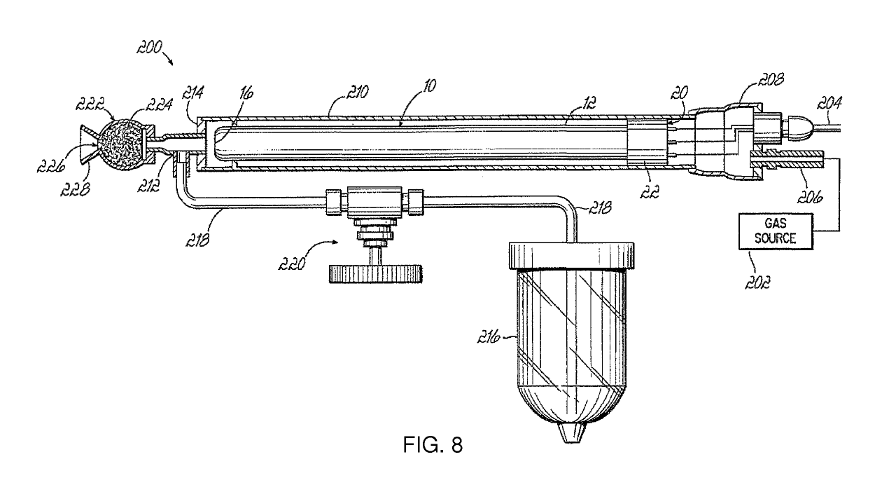

A further embodiment of an industrial heater 10 according to this invention is shown in FIG. 8 and is adapted to generate super heated steam. Traditionally, boiling water at high pressure and then heating the steam at high pressure have produced super heated steam. The embodiment of FIG. 8 provides a device where the flow of hot air over an orifice causes a super saturated steam jet. Components of the industrial heater and steam generator 200 shown in FIG. 8 that are the same or similar to corresponding components of the heater 10 as shown in FIGS. 4-6 are labeled in a similar manner. The words “superheated”, “supersaturated” and variations thereof are interchangeable. Superheated steam for the purposes of this specification is steam at less than 100° C. at 1 atmosphere or at high pressures greater than 1 atmosphere. It also encompasses H₂O in the form of gas at any temperature. Although we use the word steam to illustrate making H₂O gas or vapor we anticipate with this word any embodiment for the conversion of any fluid to a gaseous state with our apparatus and method. The word supersaturated steam is used to indicate H₂O or other materials in the form of gas at temperatures above 100° C. at pressures of about 1 atmosphere and/or higher. By supersaturated steam we also infer H₂O in the form of vapor. One objective of this aspect of this invention is to make supersaturated steam at 1 atmosphere; whereas, it normally takes high pressure to make supersaturated steam. Although we use the word steam to illustrate making H₂O gas or vapor we anticipate with this word any embodiment for the conversion of any fluid to a gaseous state with our apparatus and method. We also intend to use the words superheated and supersaturated interchangeably.

The superheated steam generator 200 includes a gas inlet source 202, which may be pressurized or unpressurized, and a power cord grip 204 proximate a gas inlet 206 of the device. A manifold housing 208 is mounted on the gas entry end of a casing 210 that is generally a right circular tube. An industrial gas heater 10 according to a variety of embodiments according to this invention such as those shown in FIGS. 4-6 is mounted within the casing 210.

Proximate the gas exit port 16 of the industrial gas heater 10, a delivery tube 212 is mounted to an end plate 214 of the casing 210. The delivery tube 212 is in communication with a fluid reservoir or cup 216 which may be a polycarbonate reservoir. The delivery tube 212 advantageously includes a venturi assembly therein. A supply or feed line 218 from the reservoir 216 is regulated by a needle valve 220, the operation of which is well known by those of ordinary skill in the art. The valve 220 may be either mechanical, electromechanical, semiconductor, nano valve, needle valve, self regulation condition by water level or any other commonly understood regulating device with or without feedback. The feed line 218 is coupled to the delivery tube 212 as shown in FIG. 8. The supply feed line 218 may be stainless steel piping or other appropriate material. The delivery tube 212 feeds into a reactor vessel 222 having a generally bulbous configuration. Contained within the reactor vessel 222 is a porous medium 224 such as steel wool or other generally non-dissolvable media; however, a dissolvable media may be utilized within the reactor vessel 222, if appropriate. The porous medium 224 may be made of metallic, ceramic, polymer, intermetallic, nano-materials, or composite materials or combinations and mixtures thereof. The porosity may be reticulated or well defined. The porosity may be even or uneven and may vary from nanometer-size to centimeter sized pores. An exit nozzle 226 is provided on the reactor vessel 222 and may include a diffuser 228.

The liquid to be heated into super saturated steam is contained within the reservoir 216 and fed to the venturi tube through the inlet pipe as regulated by the needle valve. The gas heated by the gas heater passes into the delivery or venturi tube 212 that is connected to the liquid reservoir 216. As the hot gas passes through the venturi tube 212, it draws the liquid from the reservoir 216. The liquid flow as previously stated is controlled by the needle valve 220. The liquid is atomized in the venturi tube 212 and the liquid/gas mixture enters the reactor vessel 222 where the liquid is vaporized. The unique design of the reactor vessel 222 provides for total vaporization of the liquid. The vaporized fluid exiting the reactor vessel 222 may be re-circulated through the superheated steam generator 200 and introduced into the gas inlet 202. Furthermore, the apparatus and method of this invention may produce steam by the addition of H₂O through one or both of the coils in the gas heater 10. This introduction of the H₂O may be at the inlet, outlet or in-between the gas passage and the H₂O may be added in the form of a liquid, gas or mist.

We have noted that the position of the valve 220 influences the air steam mixture. For example, at 100 ml of water in 462 seconds, a high 40% specific humidity value at 375° C. at about 1.3 cfm of hot air is generated. The relative humidity is estimated to be about 40% at this temperature assuming full compositional scale ideal gas mixing with no mixing enthalpy. Further, at 375° C., a pressure of 22 MPa (i.e., approximately 220 times atmospheric pressure) is needed to initiate condensation of the mixture. Alternatively, cooling the gas to about 110° C. at one atmosphere is required to initiate condensation. Specific humidity is defined as the mass of H₂O divided by the mass of air.

Steam temperature depends on the water valve 220 setting and air inflow setting. Typical settings at a full power of 1 kW for the gas heater to are as follows: gas at 1.45 CFM and water at 200 ml in 45 minutes yields steam air temperature of approximately 350° C. Gas at 1.4 CFM and water at 200 ml in 20 minutes yields steam air temperature of about 250° C. Further, gas at 1.8 CFM and water at 200 ml in 20 minutes yield steam air temperature of about 150° C. The above examples utilize a gas inlet temperature at approximately 30° C. and the water inlet temperature at approximately 30° C.

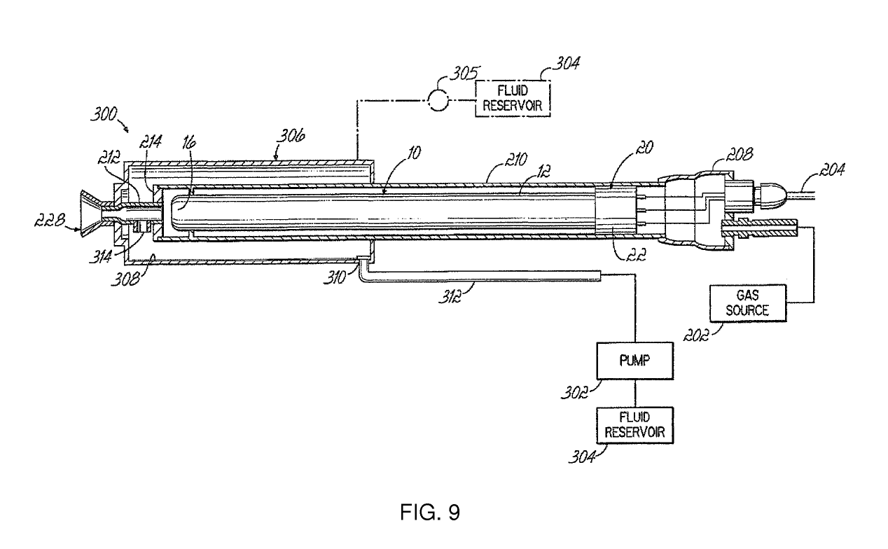

A superheated steam generator 300 in accordance with another embodiment of the invention is illustrated in FIG. 9. The superheated steam generator 300 is similar to the superheated steam generator 200, and thus only the differences between the two will be described in detail. Similar reference numerals will refer to similar features as shown in FIG. 8. In this embodiment, the use of a venturi to draw the working fluid from fluid reservoir 216, and the use of the reactor vessel 222 may be eliminated. Instead, and in one embodiment, a pump 302 may be used to actively supply the working fluid to the superheated steam generator 300 from a fluid reservoir 304. For example, the pump 302 may be a peristaltic pump having the necessary controls for selectively metering the flow rate of the working fluid (e.g., water) to the superheated steam generator 300. Such peristaltic pumps are commercially available. Other arrangements for supplying the working fluid to the superheated steam generator 300 are also within the scope of the invention. By way of example, a passive arrangement (shown in phantom in FIG. 9) may be utilized wherein the fluid reservoir 304 (e.g., water bag, cartridge, etc.) supplies the working fluid to the heater and steam generator 300 through gravity, for example, or other passive means. In such an embodiment, the reservoir 304 may include appropriate valving 305 (e.g., drip chambers, clips, etc.) for metering the flow of the working fluid to the superheated steam generator 300. Another modification to superheated steam generator 300 is the inclusion of an outer jacket housing 306 that defines a chamber 308 about at least a portion of the casing 210 having an inlet 310 for receiving the working fluid from pump 302 via a suitable conduit 312, and an outlet 314 in fluid communication with delivery tube 212. While the outer jacket housing 306 is shown adjacent the outlet side of the superheated steam generator 300, the housing 306 may be located along other portions of the heater and steam generator.

In operation, the pump 302 or other active or passive supply device supplies the working fluid from the reservoir 304 through conduit 312, through inlet 310, and into the chamber 308 defined by housing 306. The heater 10 heats the casing 210 sufficiently to preheat the working fluid contained in chamber 308 to near or at its saturation temperature (e.g., boiling point). Thus, saturated liquid, saturated vapor or both may be present in chamber 308. Similar to the previous embodiment, the fluid in chamber 308 then flows into the delivery tube 212 where it mixes with the heated gas exiting gas heater 10. The heat from the gas causes the working fluid introduced from chamber 308 to become superheated. In one embodiment, the working fluid is water and the superheated steam generator 300 generates superheated steam. Other working fluids, however, may be used in accordance with aspects of the invention as mentioned above. The end of the delivery tube 212 may include a threaded portion for coupling to various exit nozzles 228 that facilitate directing the superheated vapor-gas mixture (e.g., steam-air mixture) toward various items 230.

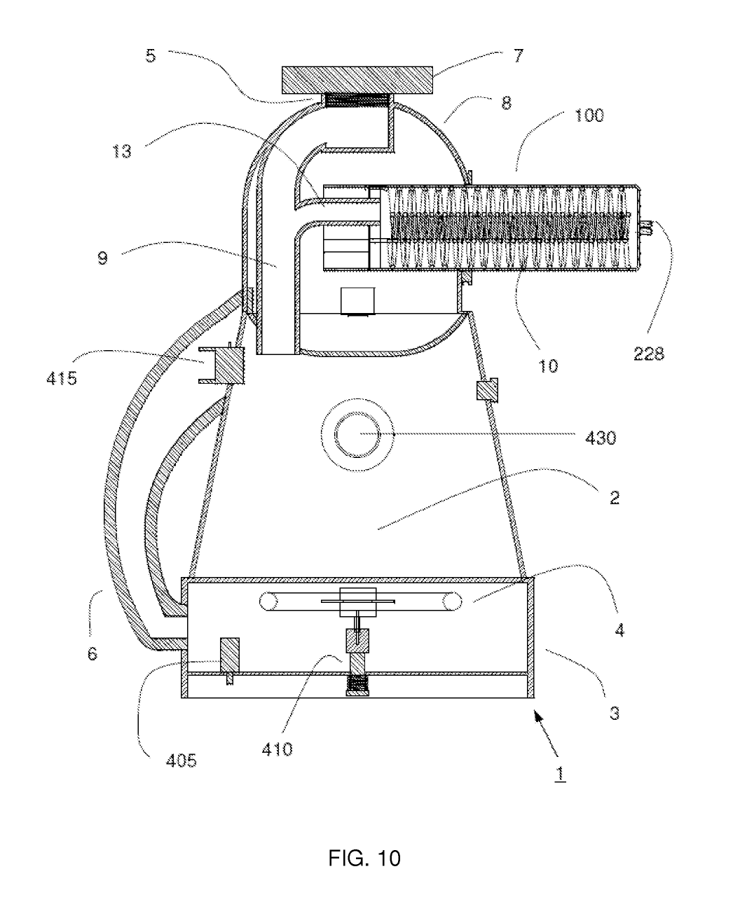

FIG. 10 shows a cross sectional view of an exemplary embodiment of the compact steamer 1 which may be handheld. The embodiment comprises a steam vessel 2, a base 3, heating coils 4, a fill opening 5, a handle 6, a screw cap 7, a neck 9 and a superheated steam generator 100. In this embodiment the superheated steam generator 100 is comprised of an industrial heater 10 comprising coil-in-coil technology as described above and a means to provide steam to the industrial heater 10 for its conversion to superheated steam. This means may comprise a neck 9 having a steam return 13 connected to the industrial heater 10 which allows passage of steam into the heater.

In operation, liquid is added into the steam vessel 2 through the fill opening 5 via the neck 9. The fill opening 5 may be threaded and sealed with a screw cap 7. The coils 4, located in base 3 are energized by an external power source thereby producing steam. The steam rises through the neck 9 and is directed to the industrial heater 10, which is also powered by an external source of electricity, through a tubular steam return 13 which is connected to, and branches from the neck 9. The industrial heater 10 and the steam return 13 comprise the superheated steam generator 100 for this embodiment. The screw cap 7 seals the steamer, forcing the steam into the steam return 13 and the industrial heater 10 where it is converted to superheated steam and expelled out nozzle 228. The steamer is kept at one atmosphere due to the unobstructed free flow of superheated steam through the industrial heater 10. Any pressure caused by the generation of steam is released immediately though the nozzle 228 leading to a very safe steamer. It is anticipated that this embodiment may also comprise a power switch 405, a safety cut-off 410, a heater switch 415 and a liquid level window 430.

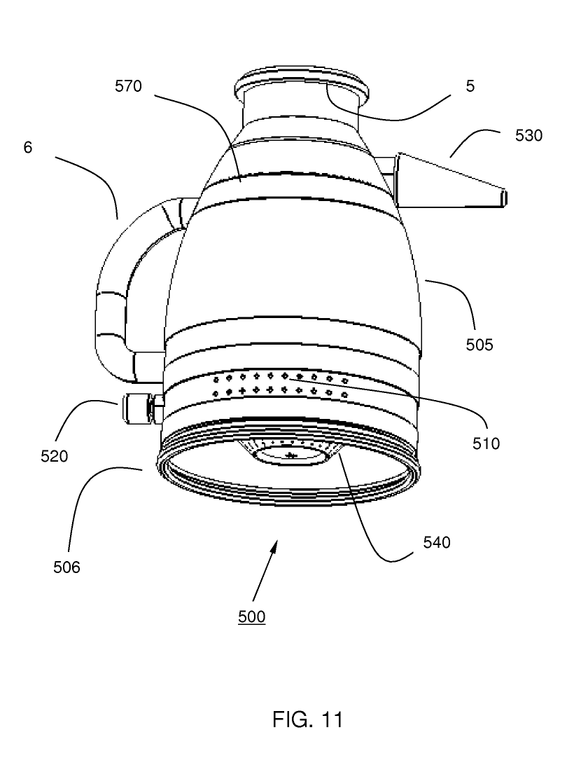

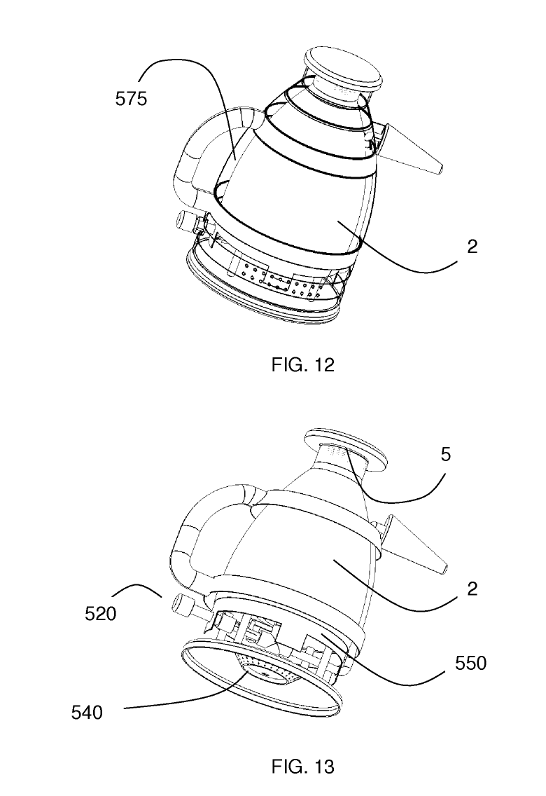

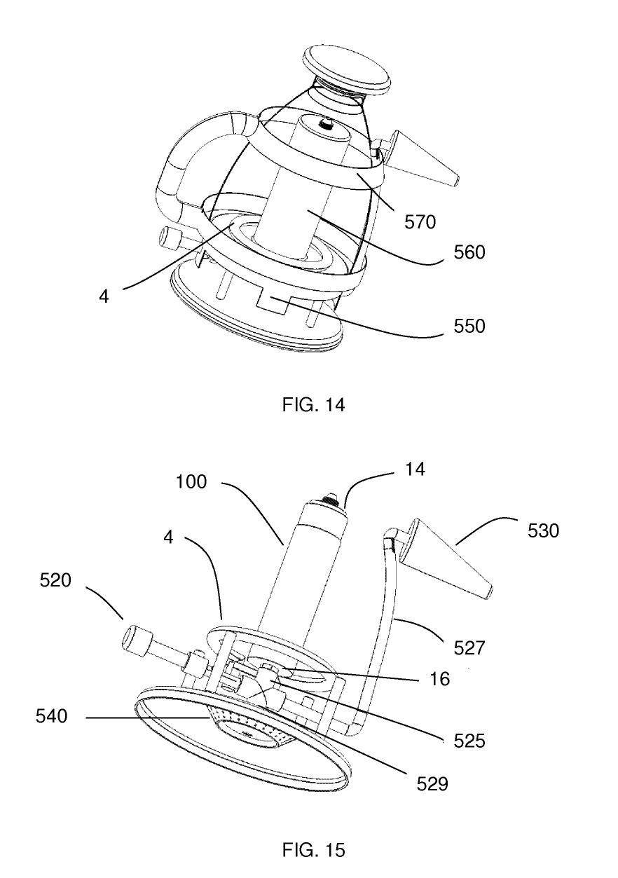

FIGS. 11-15 show a further embodiment of the compact and handheld steamer 500. The steamer 500 is intended to hold a maximum of 1 liter of water and is designed to provide either a direct concentrated stream or a gentle diffused flow of superheated steam. The steamer 500 is comprised of an outer shell 505 that may be composed of blow-molded plastic or other suitable material. The shell 505 may be bottle-shaped with a concave base 506 at the bottom of the shell 505 and a fill opening 5 at the top of the shell 505. The shell 505 is configured with cooling holes 510 which allow air to enter and provide cooling for the heating coil 4. A handle 6 is attached a side of the shell 505 at a right angle to the base 506. A fill level band 570 may be attached around the circumference of the shell 505 to indicate a level of 1 liter of water. The steamer 500 is designed so that 1 liter of liquid added to it when empty will not fill the steamer past the fill level band 570. Also contemplated are steamers with maximum volumes of more or less than 1 liter.

The shell 505 contains a frame 550 which is fabricated from sheet metal and is located in the lower third of the shell 505. Above, and supported by, the frame 550 is a hollow bottle-shaped steam vessel 2. The steam vessel 2 is the same shape as, and slightly smaller than, the shell 505 and is found inside of the shell 505 thereby delineating a uniform space 575 between the shell 505 and the vessel 2. The upper end of the vessel 2 terminates at the fill opening 5. The upper end of the vessel 2 is open and threaded to accept a screw cap 7.

A superheated steam generator 100 is positioned vertically at the center of the bottom of the vessel 2 at right angle to the bottom of the vessel. The generator 100 is also positioned so that gas entry port 14 of the generator 100 is pointed up toward the fill opening 5 and the gas exit port 16 is pointed down towards the concave base 5. A shroud 560 encases the generator but permits access for steam into the gas entry port 14 and allows steam to leave via the exit port 16. A heating coil 4 is positioned inside the vessel 2 at the vessel bottom 562. It is envisioned that the heating coil 4 could be located at the outside of the vessel bottom 562 as well. The heating coil 4 circles around the generator 100 at the vessel bottom 562. The heating coil 4 and the superheated steam generator 100 are both connected to an external electrical power source not shown.

Inside the shell 505 the frame 550 also supports a diverter valve 525 underneath the vessel 4 and connected to the exit port 16 of the generator 100. The diverter valve 525 is controlled by the diverter switch 520 which extends out through the outer shell 505. Superheated steam from the generator 100 is directed by the diverter 525 through a diffused steam line 529 to a diffused applicator located at the concave base 506. Alternatively, the diverter 525 may direct superheated steam through the direct steam line 527, located in the uniform space 575 between the vessel 4 and the outer shell 505, and out of the shell 505 to a direct applicator 530 positioned outside the outer shell opposite the handle 6. The direct steam line 527 may extend outside of the shell at various distances to meet application needs. The line 527 may be flexible or rigid. The line 527 may be configured to draw the superheated steam out of the steamer and may also provided added velocity for the projection of steam onto objects and into tight spaces. A variety of shaped applicators 530 may be positioned on the end of the line 527 depending on usage and desired application.

In operation, the compact steamer 500 is filled with a maximum of 1 liter of water via the fill opening 5 which is then sealed with the screw cap 7. The water level inside the vessel 2 is kept below the top end of the superheated steam generator 100 at the gas entry port 14. Such water level is indicated by the fill level band 570. This allows an area inside of the vessel 2 for steam to form and accumulate. The compact steamer 500 is positioned so that the superheated steam generator 100 is vertical to the ground in order to keep the water below the top of the generator 100. The correct water level can be achieved by either adding 1 liter when the compact steamer 500 is empty or by gauging the level by comparing the water level inside the vessel 2 with the fill level band 570 positioned on the shell 505.

Soon after the heating coil 4 is energized steam begins to accumulate above the water level in the vessel. At the same time, the superheated steam generator is being energized as well, possibly from the same electric source which is energizing the heating coil 4. As the steam builds it flows into the generator 100 through the gas entry port 14. In the generator 100 the steam is converted to superheated steam which flows out of the gas exit port 16. It is anticipated that a steady state of steam at 420° C. can be attained in 5-6 minutes. At this point the superheated steam will be directed by the diverter 525 through either a diffused steam line 529 or a direct steam line 527. The diffused steam line 529 is directed downwards from the diverter 525 to the diffused applicator 540 located in the concave base 506. Alternatively, the steam may be directed by the diverter 525 through the direct steam line 527 to the direct applicator 530. Direct steam line 527 may also be described as a tube through which steam may be drawn away from the generator 100 for application on remote items.

The diversion of the superheated steam allows the compact steamer 500 to be used for direct or indirect application. A gentle stream of diffused mist of superheated steam may be provided by the diffused applicator 540. The steamer could set down where needed, even on top of infestations of unwanted pests. It is envisioned that the diffused application could be extended to a larger, even room sized area by the supplying of superheated steam into a tent-like structure or under a tarpaulin or other means of covering a large area or pieces of furniture or other items needing steam treatment. A direct stream of concentrated superheated steam may be applied when using the direct applicator. Bedbugs or other insects could be reached and eliminated in hard to get at locations. With the superheated steam at 420° C. the destruction of bedbugs and other insects as well as many microbes is almost instantaneous.

The superheated steam of this and other embodiments described in this application is generated at one atmosphere. The design and construction of the compact steamer allows for any pressure created by the generation of steam to be relieved immediately by the free flow of the steam and superheated steam out of the applicators or exit nozzles. In this way safety is enhanced and costs are kept low.

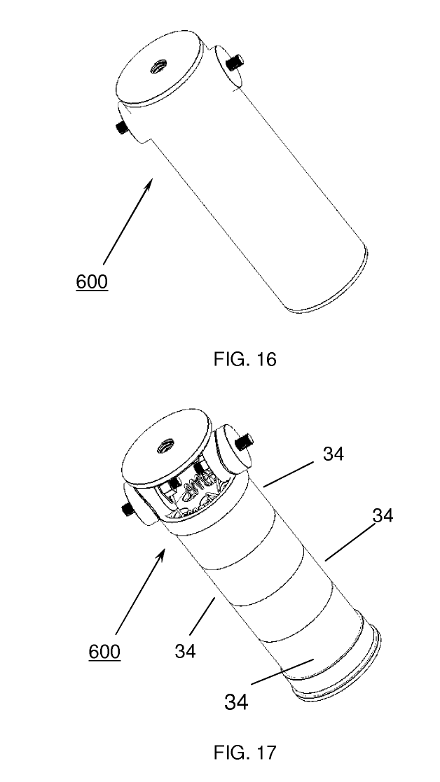

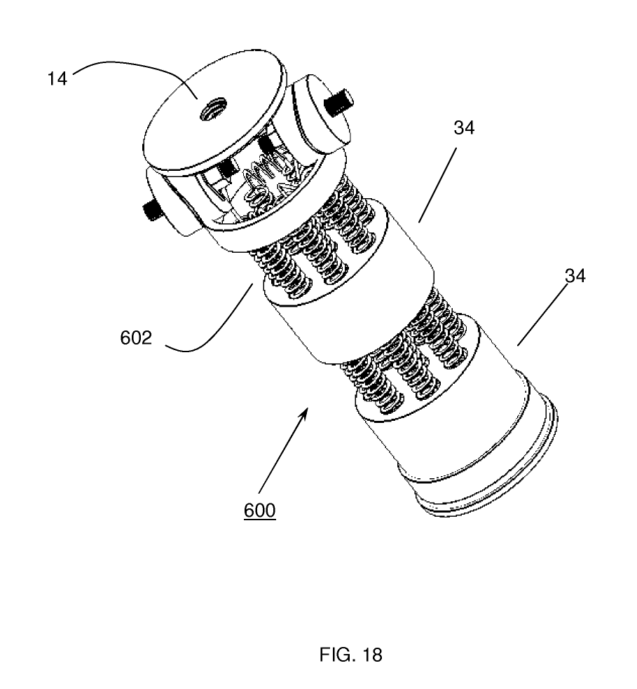

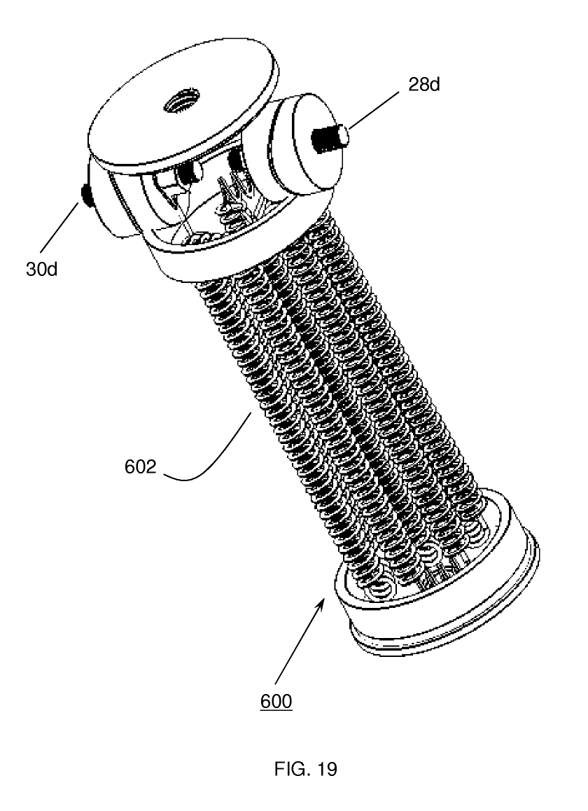

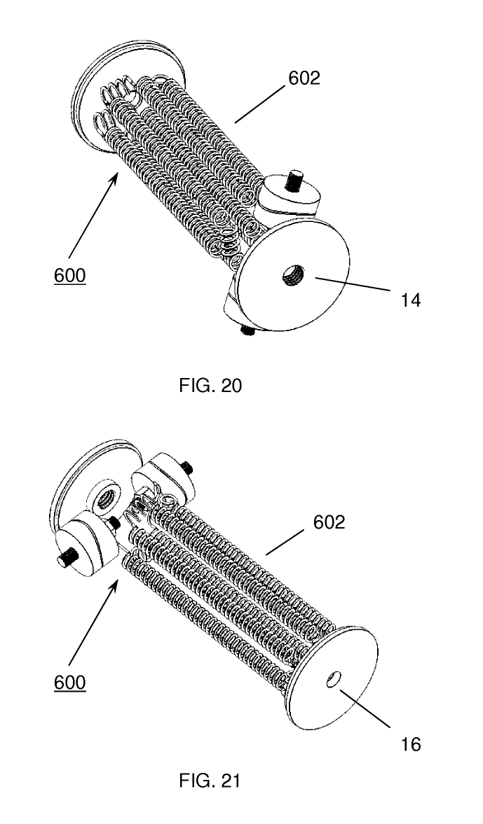

The superheated steam generator 100 of this embodiment is envisioned to be comprised of the coil-in-coil industrial heater 10 as in the embodiment of FIGS. 1 and 10. It is also anticipated that the industrial heater 10 could be replaced with a supercharger 600 shown in FIGS. 16-21.

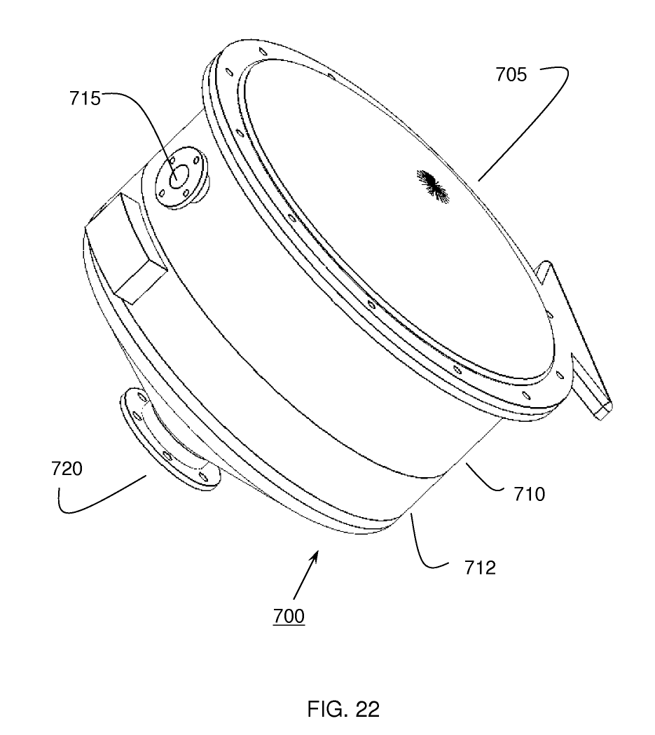

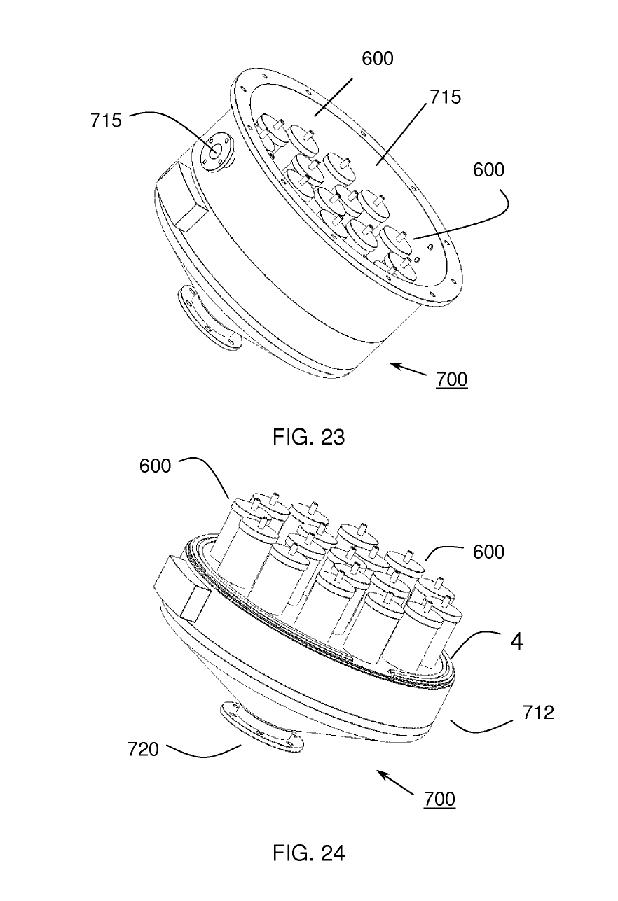

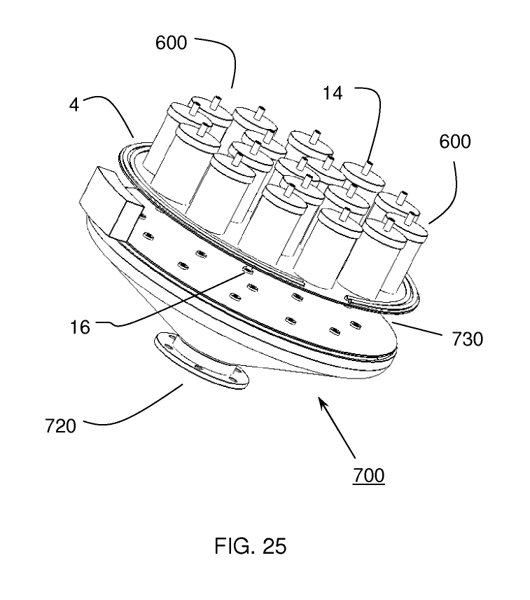

FIGS. 22-25 show an embodiment of the steam heater 700 employing multiple superchargers 600. Larger versions of this embodiment may not be handheld but are set in place to provide steam for a specific area and may be multi-watt in design and operation. This embodiment comprises a casing made up of an upper body 710 and a lower body 712. A lid 705 is positioned on top of the upper body and acts to seal an internal water chamber 715. Liquid is heated to gas within the water chamber 712 by coil 4. The gas is then passed through multiple superchargers 600 where the gas is superheated. The superheated gas is collected in the steam chamber 730, and then the superheated gas is extracted through a single steam outlet 720. In many applications water would be heated and converted to steam and then to superheated steam, but other liquids and their resulting gases are contemplated as well. As with other embodiments, this embodiment operates at one atmosphere.

In operation embodiment 700 operates in the same way as does embodiment 500 but with multiple superchargers instead of merely one. Also, in operation embodiment 700 will be positioned in a configuration where the superchargers 600 are vertical to the ground, or the lid 705 is perpendicular to the ground to keep the water or liquid level in the water chamber 715 below the tops of the superchargers 600.

Applicants envision the following features or applications in embodiments of the steam heater; thermocouples for control and over-temperature, thermal insulation where needed, a one way valve with volume flow control for water input, and gasket materials including graphite foil for sealing. Contemplated embodiments include; an 18 kW or other multiple kW steamers, a multiple boiler system with a controller, and a steamer with a 600° C. exit temperature.

Other embodiments may be equipped with the ability to change and vary the heat levels of steam produced by the steamer to destroy different species of insects that are attracted to and/or eliminated by different heat levels. Various and different nozzle shapes are envisioned for more effective and direct application of the superheated steam produced by the compact steamer. Such nozzle shapes and shapes may include, but are not limited to, divergent, flat, fan, mist, spray, knife edge or direct application configurations. Different nozzles are envisioned in order to change the energy form of the steam produced (i.e. potential and kinetic) in order to increase the effectiveness or utility of the application. Such nozzles may be constructed from adiabatic materials as well as utilizing a method to control the heat transfer from the superheated steam to the nozzle.

Still other embodiments may include a means to provide carbon dioxide (CO₂) to the fluid (the fluid may be gas, steam or liquid) and superheated steam of the steamer in order to attract bedbugs. Bedbugs are attracted to their victims by sensing the CO₂ produced through the breathing process of the victim. It has been shown that a CO₂ source will attract bedbugs and can be used as bait. As CO₂ source in the steamer could be, but not limited to, in the form of CO₂ tablets or other gas producing tablets placed in the fluid supply of the steamer. The CO₂ would be transferred to the steam, thereby attracting bedbugs. It is foreseen that other insect attracting chemicals could also be added to the fluid supply of the steamer and thus transferred to the superheated steam via gas, solid, liquid or plasma additives to the appropriate points in the steam and in the steam generation and application process. Besides pest removal, the superheated steam produced by this apparatus may be otherwise used for, but not limited to, surface and item disinfection and cleaning, bird dropping removal, food preparation and drying and the elimination of microorganisms.

It also contemplated that a series of one-way check valves and pressure chambers as disclosed in PCT/US 12/25334 could be incorporated into an embodiment of the present application to increase the pressure of the steam to be applied. Such series of valves and pressure chambers could be included within the steamer or without in a position to convert the steam produced by the steamer at one-atmosphere to pressures above one atmosphere.

It is also anticipated that mixtures of fluids, solids and/or gases may be made at various stages of the superheated steam production and application process. It is foreseen that: The fluid to be converted to a gas may be mixed with a liquid, solid or gas before it is initially heated to a gas by the coil 4. The gas thus produced may be mixed with another liquid, solid or gas within the steam vessel or chamber before it is superheated. The resulting superheated gas may be mixed with a gas, solid or liquid after it has been superheated, directly before application. One or more of these mixing operations may be performed depending on the circumstances and desired applications. A variety of gases and liquids may be chosen to achieve desired colorant, attractant, disinfectant or other attributes which are desired.

To provide safety for the operator and as a means to more effectively apply the steam, a colorant could be added to the liquid supply to aid in the sighting of the steam as it flows from the steamer. A user would then be better able to see the steam and ascertain if it is being produce and in what direction it is being projected and what surfaces it is being applied.

A further embodiment anticipates a supercharger 600 or industrial gas heater 10 placed within the tubing or piping comprising a steam line leading from the steam generator of the compact steamer or other steam or superheated steam generator to the point of application of the steam. Multiple superchargers or heaters would be placed at distances along a run of piping to keep the steam in the piping at superheated temperatures. This would keep the steam superheated and decrease if not eliminate condensation from forming in the piping as the steam cools.

The superchargers or heaters are designed to allow gas or steam to flow through them. One or more units could be placed at intervals along a run of piping. The proper number and distance could be tailored to the needs of the system and the application. The heaters could be connected in series or parallel with necessary wiring running through the steam lines as well. Previously, such piping was heated externally or insulated in attempt to keep the steam at superheated temperature. These methods only heat the steam indirectly and often ineffectively and inefficiently.

Heating the steam at intervals within the piping is much more efficient as the steam is heated directly without obstruction from the walls of the piping. Also, such a system would take up no room outside of the piping.

The above descriptions provide examples of specifics of possible embodiments of the compact steamer and should not be used to limit the scope of all possible embodiments. Thus the scope of the embodiments should not be limited by the examples and descriptions give, but should be determined from the claims and their legal equivalents.

We claim:

1. A compact steamer comprising a vessel for the initial heating of a liquid to a gas, a means to heat said liquid and at least one superheated steam generator for the heating of said gas to superheated temperatures at one atmo sphere.

2. The compact steamer of claim 1 wherein said vessel comprises a base, a sealable opening and a handle.

3. The compact steamer of claim 1 wherein said compact steamer is configured to be handheld.

4. The compact steamer of claim 1 wherein said superheated steam generator is comprised of a tubular enclosure having a gas entry port spaced from a gas exit port, an inner helical coil contained within the tubular enclosure, and an outer helical coil contained within the tubular enclosure surrounding the inner coil to define a substantially unobstructed annular space between the coils and wherein said inner helical coil and said outer helical coil are comprised of bare wire.

5. The compact steamer of claim 2 wherein said vessel and said superheated steam generator are connected by a neck, said neck being hollow, allowing for free flow of said gas from said vessel through said neck and through, and out of, said superheated steam generator.

6. The compact steamer of claim 2 wherein said means to heat said liquid comprises at least one electric heating element positioned in said base of said compact steamer.

7. The compact steamer of claim 2 further comprising a self-contained liquid supply, said self-contained liquid supply being held within said vessel and being placed within said vessel via said sealable opening.

8. The compact steamer of claim 1 further comprising an internal power supply.

9. (canceled)

10. The compact steamer of claim 1 further comprising an external power supply.

11. (canceled)

12. The compact steamer of claim 2 wherein said vessel and said handle are coated with nanoscale anti-microbial coatings.

13. The compact steamer of claim 1 wherein said superheated steam generator is capable of reaching temperatures in the range of 100° C. to 500° C.

14. (canceled)

15. (canceled)

16. The compact steamer of claim 1 further comprising an applicator, wherein said applicator allows for the direct projection of superheated steam onto an object.

17. The compact steamer of claim 1 further comprising an applicator, wherein said applicator allows for diffused projection of superheated steam onto an object or into an enclosed area.

18. The compact steamer of claim 1 wherein the superheated steam generator is further comprised of a gas exit port and a venturi tube positioned at the gas exit port.

19. The compact steamer of claim 1 further comprised of a tube positioned and configured to draw the superheated steam away from the superheated steam generator for application of the superheated steam on remote items.

20. A method for the treatment of surfaces and objects with superheated steam comprising boiling of a liquid into a gas, heating said gas to superheated temperatures at one atmosphere and applying said gas at said superheated temperature on said surfaces and said objects wherein any pressures resulting from the boiling of said liquid are relieved by a free flow of said gas.

21. The method of claim 20 wherein the boiling of a liquid into a gas, the heating of said gas to superheated temperatures at one atmosphere and the applying of said gas are accomplished with a compact steamer comprising a vessel for the initial heating of said liquid to said gas, a means to heat said liquid and a superheated steam generator at one atmosphere for the heating of said gas to superheated temperatures, wherein said vessel and said superheated steam generator are connected by a neck, said neck being hollow, allowing for free flow of said gas from said vessel through said neck and through, and out of, said superheated steam generator.

22. The method of claim 20 wherein said superheated steam generator of said compact steamer is comprised of a tubular enclosure having a gas entry port spaced from a gas exit port, an inner helical coil contained within the tubular enclosure, and an outer helical coil contained within the tubular enclosure surrounding the inner coil to define a substantially unobstructed annular space between the coils and wherein said inner helical coil and said outer helical coil are comprised of bare wire.

23. The method of claim 20 wherein the use of the superheated steam is selected from the group consisting of drying food, killing insects, eliminating microorganisms, cleaning items and disinfecting surfaces.

24. (canceled)

25. (canceled)

26. (canceled)

27. A method to maintain steam within a steam line at a superheated temperature comprising; positioning at least one heater within the steam line, powering the at least one heater to a temperature capable of keeping the steam at the superheated temperature and flowing the steam through the steam line past and through the at least one heater thereby keeping the steam at the superheated temperature.
